(19) United States
(12) Patent Application Publication
(10) Pub. No.: US 2007/0118045 A1
(43) Pub. Date: May 24, 2007

(54) Iontophoresis challenge for monitoring cardiovascular status

(75) Inventors: Morteza Naghavi, Houston, TX (US); Timothy J. O'Brien, Anoka, MN (US); Craig Jamieson, Houston, TX (US); Mark C. Johnson, Houston, TX (US)

Correspondence Address: WONG, CABELLO, LUTSCH, RUTHERFORD & BRUCCULERI,;L.L.P., 20333 SH 249, SUITE 600, HOUSTON, TX, 77070, US

(73) Assignee: Endothelix, Inc., Houston, TX (US)

(21) Appl. No.: 11/585,781

(22) Filed: Oct. 23, 2006

Related U.S. Application Data
(60) Provisional application No. 60/728,874, filed on Oct. 21, 2005.

(51) Int. Cl. A61B 5/00; A61B 6/00; A61B 5/02

(52) U.S. Cl. 600/549; 600/301; 600/500; 600/485; 600/474

(57) ABSTRACT

Methods and apparatus are provided for determining individual vascular responses by employing iontophoresis to deliver vasoactive compounds to local area and determining resultant changes in blood flow by measuring changes in skin temperature as a correlate of local blood flow. The invention further provides methods and apparatus for assessing vascular reactivity in individuals under ambulatory conditions and relating stress responses to vascular reactivity.

### CROSS REFERENCE TO RELATED APPLICATIONS

This application is claims priority under 35 USC §119 to U.S. Provisional Application No. 60/728874, filed Oct. 21, 2005, the disclosure of which is incorporated herein by reference.

### FIELD OF THE INVENTION

This invention relates methods and apparatus for assessing vascular reactivity status.

### BACKGROUND OF THE INVENTION

Without limiting the scope of the invention, its background is described in connection with monitoring the status of the vascular system, including in connection with psychosomatic stress. Cardiovascular disease (CVD), including coronary heart disease (CHD), is the leading cause of death in the United States and in most developed countries. Non-fatal manifestations of CVD require expensive hospitalization and treatment.

Studies have established that CVD occurs, and potentially can be detected, years before normal symptoms would appear. CVD is an insidious disease in that its characteristic symptoms are often manifest only at an advanced stage and under conditions of physiological stress. It is now widely known that traditional cardiovascular risk assessment such as blood testing, resting electrocardiogram (ECG) and treadmill stress tests fail to identify most individuals at risk of heart attack.

Endothelial function (EF) is becoming accepted as the most sensitive indicator of vascular function. EF has been labeled a “barometer of cardiovascular risk” and is well-recognized as the gateway to cardiovascular disease, by which many adverse factors damage the blood vessel. See Vita J A and Keaney J F Jr. “Endothelial function: a barometer for cardiovascular risk?” Circulation 106(6) (2002) 640-2. The endothelium has many important functions in maintaining the patency and integrity of the arterial system. The endothelium can reduce and inactivate toxic super-oxides which may be present in diabetics and in smokers. The endothelium is the source of nitric oxide, a local hormone that relaxes the adjacent smooth muscle cells in the media, and is a powerful vasodilator.

The endothelium regulates vascular homeostasis by elaborating a variety of paracrine factors that act locally in the blood vessel wall and lumen. Under normal conditions, these aspects of the endothelium, hereinafter referred to as “endothelial factors”, maintain normal vascular tone, blood fluidity, and limit vascular inflammation and smooth muscle cell proliferation.

When coronary risk factors are present, the endothelium may adopt a phenotype that facilitates inflammation, thrombosis, vasoconstriction, and atherosclerotic lesion formation. In human patients, the maladaptive endothelial phenotype manifests itself prior to the development of frank atherosclerosis and is associated with traditional risk factors such as hypercholesterolemia, hypertension, and diabetes mellitus. The maladaptive endothelial phenotype is further identified with emerging risk factors such as hyperhomocysteinemia, obesity, and systemic inflammation.

However, as important as assessment of endothelial function appears to be, traditional techniques for assessment of endothelial function are either invasive or require sophisticated equipment. Prior art means for estimating endothelial dysfunction include the use of cold pressure tests by invasive quantitative coronary angiography and the injection of radioactive material and subsequent tracking of radiotracers in the blood. These invasive methods are costly, inconvenient, and must be administered by highly trained medical practitioners.

Noninvasive prior art methods for measuring endothelial dysfunction include: the measurement of the percent change and the diameter of the left main trunk induced by cold pressure test with two dimensional echo cardiography, the Dundee step test, laser doppler perfusion imaging and iontophoresis, and high resolution lo-mode ultrasound. The problems and difficulties associated with the ultrasound imaging such as sensitivity to probe positioning, signal artifacts, poor repeatability, need for skilled technicians, observer dependence, observation bias, and high cost have limited the use of this invaluable test to research laboratories.

What are needed are methods and apparatus for ambulatory quantitative assessment and monitoring of the status of the vascular system including under real-life conditions including stressful situations. Also needed are methods and apparatus to measure and associate stress responses with vascular function under real-life conditions such that individuals with hidden susceptibility to pathologic vascular effects of stress can be identified.

### BRIEF SUMMARY OF THE INVENTION

The present invention provides methods and apparatus for determining individual vascular status utilizing an unobtrusive ambulatory device to monitor changes in hemodynamic parameters responsive to the introduction of a vasodilating stimulant. In one embodiment, a method of assessing vascular health in an individual is provided including continuously measuring and recording skin temperature at a test location on the individual, administering a vasostimulator compound by iontophoresis to the test location, and determining vascular health on the basis of skin temperature changes in response to the vasostimulator compound. The vasostimulator compound can be an endothelium-dependent vasostimulator compound such as acetylcholine. Alternatively, the vasostimulator compound can be an endothelium-independent vasostimulator compound, such as for example sodium nitroprusside. In one embodiment the skin temperature is measured by a thermal potential difference using a thermocouple. In other embodiments, skin temperature is measured by infrared detectors including digital infrared thermal detectors.

Suitable test locations include the forearm, wrist, forehead and finger. Optionally, additional physiologic parameters selected from the group consisting of: pulse rate, blood pressure, galvanic response, blood oxygenation, and sweating may be simultaneously measured and recorded. The use of the methods and apparatus may be further implemented as well to determine responses to induced and actual stress and for identifying individuals susceptible to detrimental effects of stress on the cardiovascular system.

In one embodiment of the invention a vascular reactivity measurement device is provided including a housing including an iontophoretic chamber adapted to store and deliver a charged vasostimulator compound through skin of a patient; and one or more temperature sensors mounted on the housing and adapted to detect changes in skin temperature in response to delivery of the vasostimulator compound. Suitable temperature sensors include thermocouples, thermopiles and infrared detectors.

In one embodiment the housing further comprises a heating element for measurement of thermal flux. In another embodiment the housing further comprises a chemical sensor for detection of compounds brought to the surface of the skin by the electric current of the iontophoresis device, including compounds involved in the nitric oxide pathway. In one embodiment, the housing is dimensioned to be affixed flat to a skin surface and can be dimensioned to be worn like a watch. In other embodiments, the housing is dimensioned to be worn as a ring on a finger.

In one embodiment, a method of vascular and/or neurovascular function measurement is provided comprising initiating monitoring of an inherent temperature of a mucosal surface; administering a vasostimulator directly to the mucosal surface; continuing to monitor any temperature change as a consequence of administration of the vasostimulator; and determining vascular and/or neurovascular responsiveness on the basis of temperature changes in response to the vasostimulator. In this embodiment, although iontophoresis can be employed, it is not required to convey the vasostimulator across the mucosa. Monitoring can be by thermal flow measurements such as with a flux gate thermal sensor, through use of contact thermal sensors, and/or with non-contact detection such as by digital infrared thermal imaging. The suitable mucosal surfaces include the eye mucosa, sublingual mucosa, intranasal mucosa, rectal mucosa, vaginal mucosal and urethral mucosa.

One embodiment of the invention provides a method for generating an ambulatory record of vascular activity by monitoring and recording blood flow differences as a consequence of one or more vasostimulant challenges wherein the challenge is applied by delivery of vasostimulants locally by iontophoresis to a site proximate to a measurement modality. In one embodiment, an iontophoresis device is used to deliver one or more vasostimulants transdermally to stimulate subcutaneous vessels and the response is measured by one or more of: local blood flow, nitric oxide related compounds (ions); redox (oxidation) and pH.

In accordance with several embodiments of the present invention, local blood flow is measured by skin temperature. In contrast to certain available technologies that employ laser Doppler flowmetry (LDF), such as the MoorLDI available from Moor Instruments, the present invention provides for determination of blood flow by measurement of skin temperature and thermal flux. Measurement by skin temperature is desirable on the basis of the simplicity of the measurement technology such that the combined device is tough, able to withstand the rigors of the ambulatory environment, and sufficiently affordable to be widely implemented. The skin temperature is a correlate of microvascular reactivity status but also appears to be a correlate of neurovascular reactivity.

The thermal monitoring microvascular reactivity based on iontophoresis challenge test can be performed by detection of skin temperature using an infrared detector. In this case, instead of using contact based thermosensors, infrared radiation sensors (such as an infrared camera) are employed for monitoring changes in temperature before, during, and after the iontophoresis challenge test.

In another embodiment, the iontophoresis device is used for measurement of local vascular response to vasostimulants administered elsewhere, such as for example an arm-cuff occlusion test (providing reactive hyperemia after ischemic challenge). In this case the iontophoresis device measures the compounds resulted from the ischemia challenge test such as anaerobic compounds. The amount of these compounds can represent the state of metabolic health and local vascular function and circulatory perfusion.

In one embodiment of the invention, the vascular stimulus administered by iontophoresis is administered during a stress test period such that the effects of stress on vascular reactivity are determined for the individual. In one embodiment, the method for determining psychological or psycho-vascular status further includes simultaneously measuring and recording additional physiologic parameters including pulse rate, blood pressure, galvanic response, sweating, core temperature, and/or skin temperature on the thoracic or truncal (abdominal) part.

In one embodiment of the invention, a method of determining an individual at risk for acute cardiovascular effects of stress is providing including measuring ambulatory stress responses in the individual; determining a vascular function status in the individual; and determining a relative risk for an acute cardiovascular effect of stress considering the ambulatory stress response in light of the vascular function status of the individual. Optionally, a cardiovascular risk factor status of the individual can be determined and considered in light of the ambulatory stress response. In one embodiment, the ambulatory stress response is measured by using iontophoresis to deliver a local vasostimulant by iontophoresis and measuring reactive blood flow by skin temperature monitoring on fingertip proximal to the site for administration of the vasostimulant when the individual is exposed to stress events. In one embodiment, the local vasostimulant is intermittently in accordance with a programmed schedule such that the vascular responses of the individual under various conditions of mental stress and physical activity are assessed. Stress can be normal situational stress of daily life or can be emulated by administration of a chemical stress inducer or by subjecting the individual to tests known to induce stress or to a virtual reality simulator. A log of stress events is correlated with the measured ambulatory stress responses.

In one embodiment, the iontophoresis device is shaped as a torus or cylinder that is flush mounted on the skin. The device has positive and negative poles to create a current. In one embodiment, the device is affixed flush with the skin using a wristband or armband.

In one embodiment, the device further incorporates thermocouples or thermopiles as temperature sensors, either as a ring around the perimeter or distributed equally on the perimeter of the device. The device is provided with or in fluid communications with a reservoir or chamber for storage of a compound (such as a vasostimulant compound, e.g. acetylcholine). When administration is desired, the compound is diffused into the skin by way of a created electrical current or circuit induced by the device which carries charged ions of the desired compound into a subdermal location. The diffused compounds affect physiological processes. For example, introduction of a vasodilator such as acetylcholine mediates increase in nitric oxide production. The ability of the microvasculature to respond by degrees of dilation is indicative of the relative health of the microvasculature. In one embodiment the iontophoresis device is used to deliver a neurostimulator or neurovascular stimulator and then measures the response by thermal monitoring.

In one embodiment of the invention, a method of determining vascular and or neurovascular reactivity is implemented by an ambulatory device for measuring vascular function having at least one finger mounted blood flow monitor in electrical communication with a control unit, and at least one iontophoresis device in electrical communication with the control unit, wherein the control unit is adapted to continuously measure and record data from finger mounted blood flow monitors as well as instruct delivery of vasostimulators by iontophoresis. In one embodiment, the apparatus further includes an ambient temperature sensor. In one embodiment the skin blood flow measurement is by an infrared imager.

In one embodiment of the invention, an ambulatory device for measuring vascular function is provided including a cuff dimensioned to be worn on a finger, the cuff including an iontophoresis modality that is able to deliver a vasostimulant compound to the local environment of the finger, and a temperature sensor for measuring blood flow to a fingertip distal to the cuff.

### BRIEF DESCRIPTION OF THE DRAWINGS

FIG. 1 graphically depicts the analyzed parameters from DTM data points from a finger on an arm subject to reactive hyperemia (black diamonds). Hypothetical data from a contralateral control finger also shown for purposes of comparison (in grey circles). FIG. 1B schematically depicts various diseases that involve endothelial dysfunction.

FIG. 2 A) depicts by a reactive hyperemia response by video thermography and by DTM. An example of a fingertip thermal response (DTM) is depicted in B), while a graphic depiction of variations in vascular reactivity is depicted in C). FIG. 2 D) depicts results from clinical studies showing the correlation with DTM results and the Framingham Risk Score. FIG. 2 E) shows the formula used to calculate TR as depicted in D).

FIGS. 3 A-K depict a number of different iontophoresis embodiments.

FIG. 4A depicts a wrist watch type iontophoresis device while FIG. 4B depicts one such iontophoresis device affixed to an arm of a patient.

FIG. 5A depicts an embodiment of a ring embodiment of an iontophoresis device in relation to a finger tip temperature monitor. FIG. 5B depicts one position for a controller of a ring based iontophoresis device, while FIG. 5C provides a different perspective view of the same embodiment as FIG. 5B.

FIG. 6A depicts a dorsal view of an embodiment showing placement of a wrist and/or finger pressure cuff and an iontophoresis based detector in addition to a fingertip temperature monitor. FIG. 6B depicts a ventral view including a pulse detector over the radial arterial.

### DESCRIPTION OF THE INVENTION

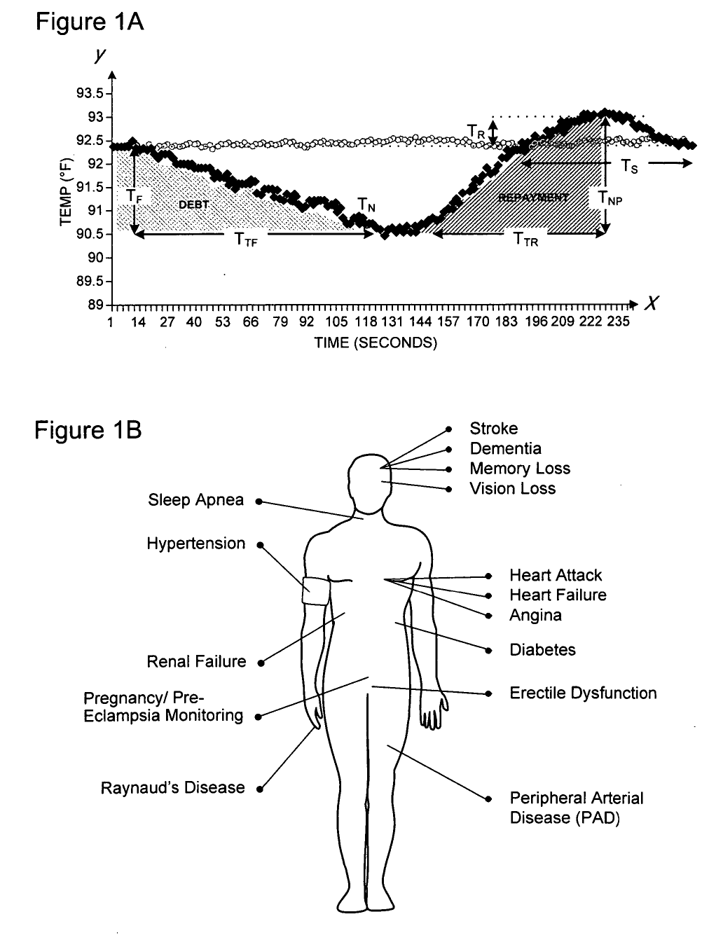

All of the blood vessels in the body are lined by a single layer of cells known as the vascular endothelium. Endothelial dysfunction causes impaired vascular reactivity, compounds the adverse effects of inflammatory factors, and underlies a variety of vascular and non-vascular diseases, particularly heart attack and stroke. Certain of the diseases associated with endothelial dysfunction are depicted graphically in FIG. 1B. Endothelial dysfunction is correlated with several risk factors, including familial hypercholesterolemia, smoking, diabetes mellitus, and hyperhomocysteinemia. In addition, repeated exposure to high levels of physical and, particularly, psychological stress, and sustained exposure to low levels of stress, both of which are experienced during active duty and high stress jobs, may impair endothelial function acutely, and cumulatively impair cardiovascular health in the longer term.

Endothelial function can be evaluated by various different approaches, including: measurement of structural characteristics of the vascular wall, e.g. intima media thickness, compliance, distensibility, and remodeling indexes; measurement of soluble endothelial markers including von Willebrandt factor, plasminogen activator, inhibitor complex thrombomodulin adhesion molecules, and nitric oxides; and measurement of endothelium-dependent regulation of vascular tone. See Kelm M. “Flow-mediated dilatation in human circulation: diagnostic and therapeutic aspects” Am J Physiol Heart Circ Physiol 282 (2002) H1-H5.

Endothelium-dependent vasodilation as a measure of endothelial function can be determined by invasive vasomotor techniques including quantitative coronary angiography and strain gauge plethysmography of the forearm with intra-arterial acetylcholine challenge. Due to the invasive nature of these methods, brachial artery flow-mediated dilation (FMD) measurement by high-resolution ultrasonography has been alternatively accepted as a research tool, albeit highly technical, for the examination of endothelial function. See Sorensen K E, et al. “Non-invasive measurement of human endothelium dependent arterial responses: accuracy and reproducibility” Br Heart J 74 (1995) 247-253.

Brachial artery flow-mediated dilation (FMD) measurement by high-resolution ultrasonography utilizes the phenomena of reactive hyperemia. Reactive hyperemia is defined as hyperemia, or an increase in the quantity of blood flow to a body part, resulting from the restoration of its temporarily blocked blood flow. When blood flow is temporarily blocked, tissue downstream to the blockage becomes ischemic. Ischemia refers to a shortage of blood supply, and thus oxygen, to a tissue. When flow is restored, the endothelium lining the macrovasculature (large vessels such as the brachial and radial arteries) is subject to a large, transient shear stress. In partial response to the shear stress, the endothelium of the macrovasculature normally mediates a vasodilatory response known as flow-mediated dilatation (FMD). The vasodilatory response to shear stress is mediated by several vasodilators released by the endothelium, including nitric oxide (NO), prostaglandins (PGI₂) and endothelium-derived hyperpolarizing factor (EDHF), among others. A small FMD response is interpreted as indicating endothelial dysfunction and an associated increased risk of vascular disease or cardiac events. See Pyke K E and Tschakovsky M E “The relationship between shear stress and flow-mediated dilatation: implications for the assessment of endothelial function” J Physiol 568(2) (2005) 357-9.

Induction of reactive hyperemia is well-established in clinical research as a means to evaluate vascular health and in particular endothelial function. Typically, a reactive hyperemia procedure is implemented by occluding arterial blood flow briefly (2-5 minutes, depending on the specific protocol) in the arm, by supra-systolic inflation of a standard sphygmomanometer cuff, then releasing it rapidly to stimulate an increase in blood flow to the arm and hand. Reactive hyperemia has been classically measured by high-resolution ultrasound imaging of the brachial artery during and after arm-cuff occlusion. However, the technical difficulties of ultrasound imaging have limited the use of this test to research laboratories. This method is clearly unsuitable to widespread adoption of reactive hyperemia as a test of vascular function. The method is simply inapplicable to evaluation of endothelial function in the context of real life stress inducers.

In addition, ischemia induces changes in the microvasculature including dilation of the vessels in an attempt to achieve increased perfusion. Failure of the microvasculature to dilate in this compensatory fashion is a feature of poor vascular function. In one embodiment of the present invention, in lieu of vascular stress induced by ischemia, vascular stress is emulated by the administration of chemical vasoactive substances by iontophoresis. Examples of vasoactive substances include acetylcholine chloride (for example 1% ACh, which is thought to be an endothelium-dependent vasoactive substance) and sodium nitroprusside (for example 1% NaNP, which is thought to be an endothelium-independent vasoactive substance). Delivery of such agents by iontophoresis has been described such as by M E Anderson, et al. “Digital iontophoresis of vasoactive substances as measured by laser Doppler imaging—a non-invasive technique by which to measure microvascular dysfunction in Raynaud's phenomenon” Rheumatology 2004 43(8):986-991. However, in contrast to prior art measurement of vascular response by laser Doppler imagery or flowmetry (LDF), the present inventors utilize thermal monitoring for measurement of blood flow.

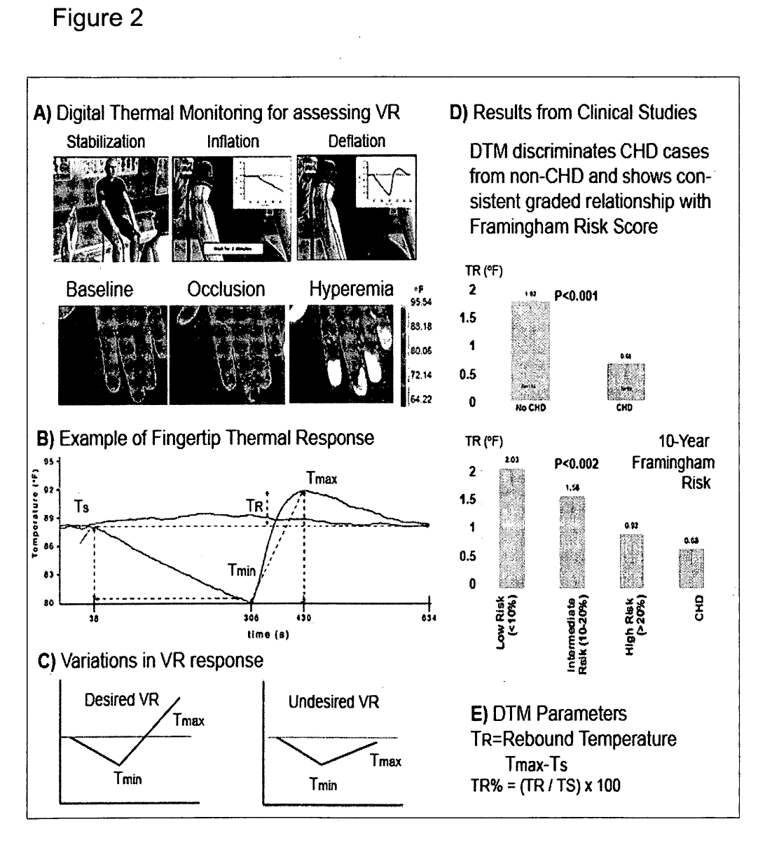

Thermal Monitoring: Certain of the present inventors have previously developed novel methods and apparatus to determine the vascular reactivity based on a measured response of the microvasculature to ischemia by continuously monitoring skin temperature on a digit distal (downstream) to an occluded arterial flow. Temperature is monitoring beginning with establishment of a baseline temperature followed by monitoring through and after administration of a vascular stress such as occlusion of the brachial artery by inflation of a blood pressure cuff. This principal and technique has been termed Digital Thermal Monitoring (DTM). See WO 05/18516, the disclosure of which is incorporated herein by reference. DTM is typically implemented by measuring temperature at the fingertips before, during and after a vasostimulus induced by transient arm-cuff occlusion and subsequent release. A normal reactive hyperemia response, i.e. increased blood flow after occlusion, is manifest by increased skin temperature over the baseline temperature established prior to occlusion. FIG. 2A depicts the steps of a DTM assessment and shows, in the bottom panel, a thermographic record of the cooling of the hand and fingers as a consequence of arm-cuff occlusion as well as the rebound temperature after release of the cuff that exceeds that of baseline in an individual with a good vascular response. Since endothelial function is a systemic property, a localized measurement in a readily accessible location of the human body (such as the digits) can provide an accurate assessment of vascular health in physiologically critical locations such as the coronary arteries. DTM is thus being developed as a new surrogate for endothelial function monitoring that is non-invasive, operator-independent (observer-independent) and is sufficiently straightforward to be readily implemented across the population to assess individual vascular function. Preliminary studies, as described below, have shown that DTM can discriminate individuals with established CHD or high risk of future CHD (as measured by Framingham Risk Score) from normal and low-risk individuals.

A pilot study was performed with the aim of evaluating the potential clinical utility in cardiovascular risk stratification of DTM. Reactive hyperemia is induced through transient inflation of a cuff placed on the arm. Skin temperature is detected by temperature sensor placed on a finger. Temperature sensor is placed on a respective finger on the contralateral hand as an internal control. In the pilot study described herein, the temperature sensor employed was a thermocouple. However, other temperature sensors might be alternatively employed in the implementation of DTM, including Resistance Temperature Detectors (RTM), thermisters, thermopiles or integrated circuit (IC) detectors.

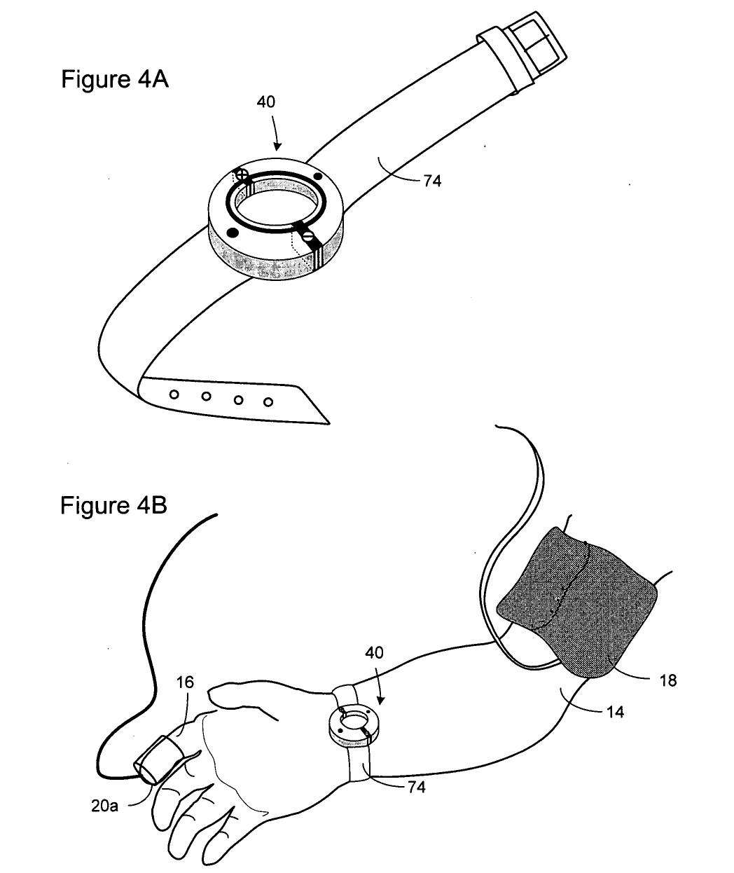

DTM assessment is conducted generally as follows. In a standard controlled setting, the subject is seated and a cuff, such as cuff 18 depicted in FIG. 4B is placed on one arm 14. Temperature probe 20a is placed on the index finger of the cuffed arm and an identical temperature probe is placed on the index finger of the contralateral arm. Baseline temperature data is continuously recorded for an equilibration period, for example three minutes. The cuff 18 is inflated rapidly to 200 mm Hg or 50 mm Hg above systolic blood pressure and the pressure is retained at this level for 2 to 3 minutes. During this period skin temperature falls on the fingertip of the occluded arm. After 2 to 3 minutes, the cuff is rapidly deflated and the skin temperature rapidly rises as blood returns to hand and fingers. Temperature is recorded for another 3 minutes after the cuff is deflated and the data from both fingers is captured and displayed by a computer. The following primary parameters are calculated as depicted in part in FIG. 1A.

Measures reflecting the ischemic stimulus/thermal debt:

| | |
|---|---|
| T_{S} | Starting fingertip temperature |
| Tₘᵢₙ | (Nadir (N)) Lowest temperature observed after cuff inflation |
| TF | Temperature Fall, T_{S} − Tₘᵢₙ |
| TTF | Time from cuff release to TF (tₘᵢₙ − tᵢ) |
| tᵢ | Time when the initial temperature was recorded |
| tₘᵢₙ | Time taken to attain Tₘᵢₙ |
| tₘₐₓ | Time to attain maximum temperature |
| t_{f} | Time to attain the equilibrium temperature (final temperature). |

Parameters reflecting thermal recovery/vascular reactivity:

| | |
|---|---|
| Tₘₐₓ | Highest temperature observed after cuff deflation |
| TR | Tₘₐₓ − Tₛ (temperature recovery/rebound) |
| NP | Nadir-to-Peak, Tₘₐₓ − Tₘᵢₙ |
| TTR | Time from cuff release to TR, (tₘₐₓ − tₘᵢₙ) |
| Slope | Slope of temperature recovery = NP/(TTR) |
| AUC | Area under the temperature-time curve |

TR and NP indicate the vasodilatory capacity of the vascular bed (small arteries and micro-vessels) and subsequent hyperemia induced brachial artery dilation. TR and NP indicate the vasodilatory capacity of the vascular bed (small arteries and micro-vessels) and subsequent hyperemia induced brachial artery dilation. TR specifically denotes the ability of the arterial bed to compensate for the duration of the ischemia and to create an overflow (hyperemia) above the baseline level. Given a good vasodilatory response and constant room temperature one would expect a positive TR. The higher the TR, the higher the vasodilatory response of the arterial bed. TR close to zero indicates a lack of strong vasodilatory response and negative TR is likely to represent a vasoconstrictive response. NP and TR largely overlap and both show similar information with TR being a more sensitive marker of overflow (hyperemia response) and NP showing additional factors that affect TF (such as neuroregulatory effect and basal metabolic rate). Factors as TTF, TTR and area under the curve are expected to provide additional insights into the response to the ischemia challenge test.

In preliminary studies, several parameters including TF, TR, NP, TTR, TTF were measured. These parameters were correlated against two standard methods of estimating blood flow changes in the forearm: flow-mediated dilatation of the brachial artery, and strain-gauge plethysmography, both during reactive hyperemia in apparently healthy volunteers. In one study, DTM results were compared against Framingham Risk Estimation (FRE) in a community setting. 133 subjects, responding to a local newspaper advertisement, gave informed consent to participate in this study. Subjects agreed to disclose limited medical information regarding any history of cardiovascular disease and cardiovascular risk factors, to a finger stick blood draw for non-fasting lipid profile measurement, and to undergo DTM on up to 3 occasions. Subjects fasted overnight and refrained from smoking, alcohol or caffeine ingestion and use of any vasoactive medications on the day of the testing in both protocols. Subjects remained seated, with the forearms supported at knee level. VENDYS™ DTM probes were affixed to the index finger of each hand as previously described.

In these preliminary studies, DTM appeared to complement FRE in distinguishing between cohorts with and without self-reported CVD. FIGS. 2A-E depict examples and results of DTM assessments of endothelial function. FIG. 2A depicts Digital Thermal Measurement (DTM) response during and after brachial artery occlusion, the thermographs indicate temperature change during the procedure. FIG. 2B depicts fingertip temperature variation recorded with VENDYS system during VR studies for occluded and not occluded hand. FIG. 2C graphically depicts variations in thermal response observed in volunteers. FIGS. 2D & E summarize results from clinical studies conducted to assess the predictive value of DTM in CVD. DTM was shown not only to correlate the FRE but offered advantages over prior techniques including: 1) low cost, 2) high sensitivity (with good specificity), 3) ease of use as a self-contained unit, and 4) reproducibility of diagnostics across a subject sample. One embodiment of the present invention now provides novel methods and apparatus that utilize the combination of iontophoresis to simulate vascular stress with DTM for ambulatory quantitative monitoring of vascular function. In addition, the methods and apparatus can be utilized to identify individuals with hidden susceptibility to pathologic vascular effects of stress.

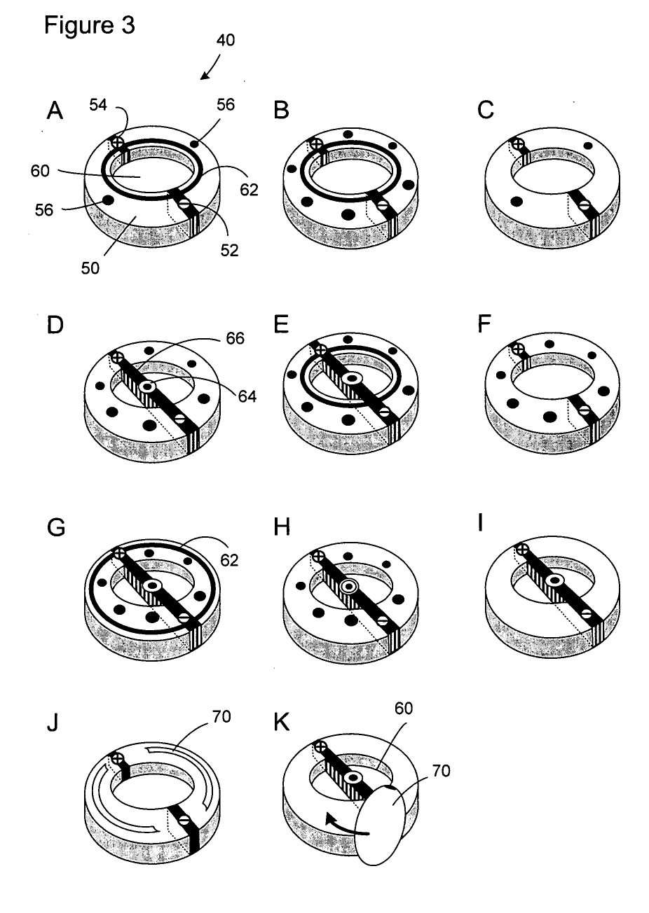

Iontophoresis Apparatus: Several exemplary embodiments of iontophoresis delivery and detector devices are depicted in FIG. 3 A-K. In the embodiments depicted in FIGS. 3A-K the iontophoresis device 40 is shaped as a torus or cylinder that provides a containment ring 50 for included instrumentalities. The device has poles (positive 54 and negative 52) to create a current.

In one embodiment of the device, temperature sensors are added to the iontophoresis containment ring 50. The temperature sensors can be provided as a ring around the skin surface of the device or can be distributed around the perimeter of the device. Examples of suitable temperature sensors include thermopiles or thermocouples 56 situated around the perimeter of the containment ring 50. The number of temperature sensors can be varied as depicted in FIGS. 3B and C.

In one embodiment, the device has a reservoir 60 in the center where a vasoactive compound such as acetyl choline (Ach) can be placed or injected into the device for later delivery. When delivery is desired, the compound is diffused into the skin by way of creating an electrical current or circuit across the device with carries the charged compound into the skin to a sub-dermal location. In the case of ACh, introduction of the vasodilator mediated increases in nitric oxide production with resulting vasodilation in normal individuals. The increased vasodilation is detected and quantitated by increased skin temperature.

In alternate embodiments, such as depicted in FIG. 3E, containment ring 50 includes a circular heating element 62 placed on the skin surface and may include a central temperature sensor 64 mounted on a support bar 66 spanning the containment ring 50. In certain embodiments, the central tempaerature sensor is an infrared sensor. The heating element is employed to heat the area while the heat sensors are used to measure changes in the spread of the induced temperature which is affected by, and is thus a measure of, local blood flow. The heating element also provides a measure of vasodilation capacity as the heat would normally stimulate vasodilation.

In other embodiment depicted in FIG. 3J, the containment ring 50 includes chemical sensor elements 70 adapted for measurement of substances such as those involved in the nitric oxide cascade, pH and redox related chemicals in the skin. As depicted in FIG. 3K, the device reservoir 60 may be provided with a cover 72 although various chambers and sealing mechanisms would be readily apparent to one of skill in the art, including for example external plug-in bladders.

In one embodiment, nitric oxide (NO) production/release is measured following delivery of a vasodilator by iontophoresis. In one embodiment the NO is estimated from measurement of the enzymatic process of NO reduction-oxidation (Redox). In other embodiments, NO is drawn to the surface and overproduction cause by diffusion of the vasodilator compound is measured. In one embodiment, induced nitric oxide is distinguished from preexisting NO such as that produced by macrophages. By monitoring baseline NO production prior to delivery of ACh by iontophoresis.

As depicted in FIGS. 4A and B, one embodiment of the device 40 can be affixed to the skin using a wrist/armband 74, so that the device is flush with the skin. In other embodiments, such as depicted in FIG. 4B, vasostimulation can be locally administered by iontophoresis or can be induced by cuff occlusion using a blood pressure cuff 18 or by systemic administration of drugs such as by sublingual, oral or inhalation administration. Other types of vasostimulation such as vagal stimulation or deep inhalation may be employed with the iontophoresis device employed in detection of nitric oxide metabolites, pH and redox pathway compounds.

As depicted in FIG. 4B, the iontophoresis methodology by be using in conjunction with DTM measurements made in the context of reactive hyperemia where it is desired to include drug induced endothelial dependent and independent assessments.

Stress Effects and the Vascular System: EF is impaired in the presence of physiologic and psychological stress. Endothelial dysfunction causes impaired vascular reactivity, compounds the adverse effects of inflammatory factors, and underlies a variety of vascular and non-vascular diseases, particularly heart attack and stroke. Conversely, EF improves with positive psychological stimuli. Thus, EF not only predicts risk, but can also parallel changes in response to therapy (pharmacologic and non-pharmacologic) and to alterations in risk factors. Psychological factors such as stress, anxiety, and depression show significant correlations with measurable physiological parameters (such as blood glucose levels, peripheral body temperature, and risk factors for cardiovascular disease). Stress also results in the secretion of cortisol which affects the blood sugar levels (abnormal levels can lead to diabetes), immune responses, and can also elicit inflammatory responses.

The relation between stress and temperature can be understood as follows. The cardiovascular mechanisms that regulate skin temperature in the hands and feet are closely linked with the activity of the sympathetic division of the autonomic nervous system. Upon activation of this system, the smooth muscles surrounding the blood vessels under the skin surface vasoconstrict, resulting in decreased blood flow to the capillaries and capillary beds (body tissue) near the skin surface. Under stress, blood flow through the peripheral capillaries and tissues near the skin surface decreases, and the temperature of the skin decreases. To achieve homeostasis (i.e. return to unstressed state), there is an increase in skin temperature as a result of vasodilatation, or relaxation of the smooth muscles surrounding the peripheral blood vessels. Vasodilatation is usually accompanied by a relaxation of sympathetic activity. There is generally an interval of several seconds between vasodilatation and skin temperature increase, because a certain time period must elapse while the increased amount of blood flows into the capillaries and tissues.

Individualized Ambulatory Assessment of Stress Reactions: Psychological stress and subclinical cardiovascular disease (CVD) interact lethally in certain individuals. In animals, including humans, acute psychological stress induces a defense reaction mediated by increased sympathetic nerve activity which in turn elicits the hemodynamic responses of increased heart rate, cardiac output, mean arterial pressure, which together with decreased renal blood flow, result in increased blood flow to the skeletal muscle of the limbs. However, in susceptible individuals, these hemodynamic responses are exaggerated and may trigger acute adverse cardiac events. Chronic effects are also implicated as stress amplifies the interaction between risk factors for atherosclerosis and vascular endothelial dysfunction. Given inter-individual differences in susceptibility, typically subtle and asymptomatic short term CV effects, and the lack of adequate methods to quantify cumulative stress exposure, it has been heretofore impossible to accurately identify those individuals at highest risk of stress-dependent CVD, including life-threatening CV events. The present inventors have developed methods and apparatus able to provide individualized assessment of stress reactions that is able to isolate the stress response from the confounding variables of general physical and environmental condition.

One embodiment of the present invention relies on continuous measurement of blood flow at anatomic locations with maximum sympathetic nervous system effects, such as the fingertip, relative to blood flow at anatomic locations with minimum sympathetic nervous system effects in order to provide a catalogue of neurovascular responses in a given individual. By continuous measurement, it is meant a series of repeated closely spaced measurements over a test period. The test period might be during the duration of a discrete administered stress test or series of tests or might be over a longer duration such as a period of hours or days.

In order for stress responses and vascular function to be assessed in the context of real life situations, including stress situations, miniaturized wearable devices are required that do not interfere with real-life activities and are able to isolate and identify neurovascular stress responses. By providing a single device that is able to both induce a vascular response by iontophoretic delivery of a vasoactive compound and record the response by local skin temperature, an extremely compact system is generated wherein the device itself does not affect the measurement by virtue of diverting the attention of the subject to bulky or instrusive equipment. Thus, the present invention is able to identify those individuals for whom intervention is medically indicated without the device itself skewing the results.

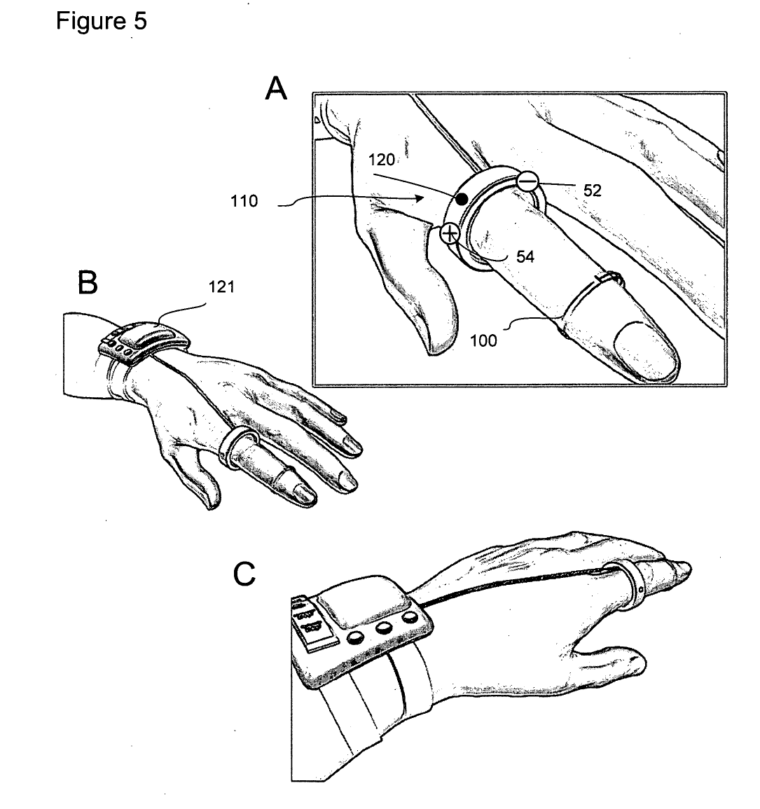

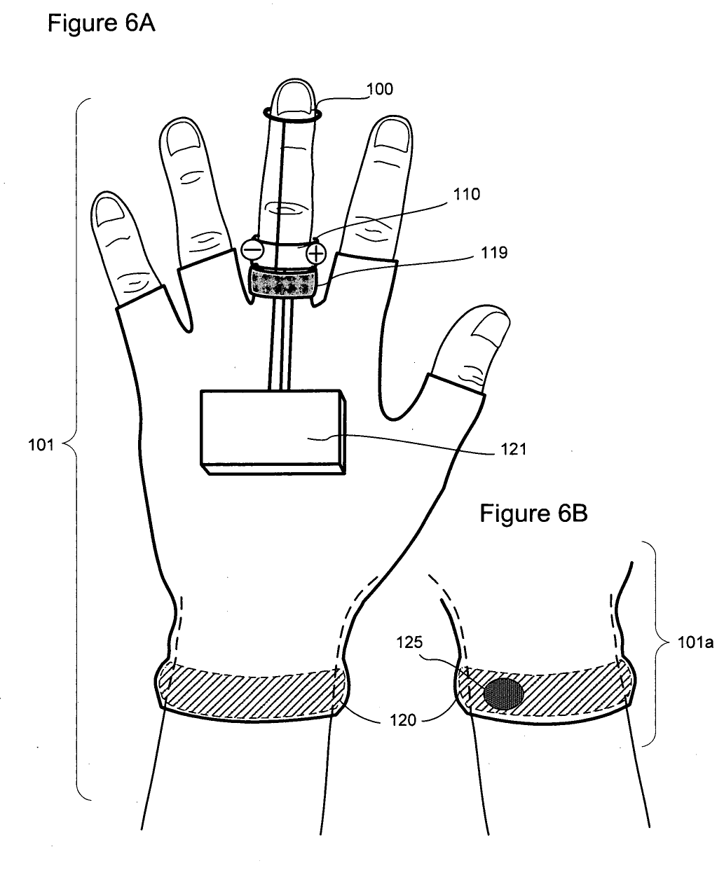

In one embodiment of the invention, a chosen location with maximum sympathetic nervous system effects is the finger. In one embodiment, as depicted in FIG. 5a, vascular reactivity is measured by a ring based iontophoresis device 110, which includes positive pole 54 and negative pole 52. A delivery port or permeable membrane (not shown) is disposed on the skin touching internal face of the iontophoresis ring 110. A refillable chamber (not shown) is disposed in the interior of the ring 110 and can be refilled through port 120. Temperature can be measured by temperature sensors disposed on the internal skin surface of the ring (not shown) or may be measured by a slightly spaced apart finger tip mounted temperature sensor 100. As depicted in FIGS. 4 B and C, the iontophoresis device may include a wrist mounted controller 121. A dorsal depiction of one such embodiment is provided by view 101 on FIG. 6. A ventral view 101a depicted in FIG. 6B, shows an optional wrist inflation cuff 120 as well pulse sensor 125. In one alternative embodiment, as depicted in FIG. 6A, a finger based inflation cuff 119 is provided as well as an iontophoresis delivery/detector device 110. A single finger sensor or a plurality of finger and palm sensors may be variously employed.

The present invention also provides a solution for obtaining accurate measurements of vascular function that discriminate between endothelial dependent versus endothelial independent vascular reactivity responses by virtue of the ability to deliver different agents effecting these disparate responses by iontophoresis. The inclusion of temperature measurement to existing iontophoresis modalities provides a simplified and highly accurate modality.

The embodiment depicted in FIG. 6 provides an optional glove assembly which optionally proves a pulse sensor 125, mounted over the radial artery. One example of a suitable pulse sensor is an oscillometric pressure sensor. The glove also optionally includes one or more of an ambient temperature sensor and a galvanic skin response (i.e., electro dermal response-EDR) monitor. Each of the functional elements of the glove is in electrical communication with a controller 121 mounted on the glove, such as on the back of the hand of the glove or on the top of the wrist. The glove can also include cuff inflation and deflation (tools) mechanisms 120 to occlude the blood flow (at wrist or elsewhere) for reactive hyperemia and vascular reactivity testing.

In one embodiment of the invention, a miniaturized device is employed to continuously measure and provide for recording of skin and ambient temperature. Because ambient temperature is also recorded, the skin temperature is provided with a contemporaneous reference. In one embodiment a method is provided for determine an individual's reaction of to induced stress. Temperature recording begins, including baseline temperature recordings. Stress monitoring by continuous skin temperature recording is combined with real and induced stress situation to provide individual assessments of stress responses. Under stress, blood flow through the peripheral capillaries and tissues near the skin surface decreases, and the temperature of the skin decreases. To achieve homeostasis (i.e. return to unstressed state), there is an increase in skin temperature as a result of vasodilatation, or relaxation of the smooth muscles surrounding the peripheral blood vessels. Vasodilatation is usually accompanied by a relaxation of sympathetic activity. A vasoconstrictive response induced by a sufficiently stressful situation is normal and maybe desirable. However, a vasoconstrictive response to a condition that should not evoke a profound stress response is undesirable. Furthermore, the intensity and duration of the response may indicate and inappropriate stress response. In one embodiment of the present invention, ambulatory blood flow monitoring by continuous skin temperature measurement is employed to identify dangerous stress responses including those where a vascular stimulus, simulated by iontophoretic release of vasoactive compounds, acts synergistically with the stress response to result in altered vascular reactivity.

In another embodiment, vascular reactivity is assessed during real-life activities by utilizing the finger or wrist based iontophoretic vascular stimulation c to measure vascular reactivity by DTM and/or fingertip arterial tonometry (for example using a device available from Itamar Medical). The device is worn in ordinary conditions, normally considered to be non-stressful, to establish a “normal” individual vascular reactivity profile. The individual is then subject to various stressful conditions to determine that individual's vascular reactivity under stress.

Device functionality is briefly described below, elaborating on the physical operating principles. Upon activation, the occluding band first compresses the artery in the finger, causing ischemia (i.e. interruption in the flow of blood to the finger tips). After a pre-set or programmable occlusion time, the finger tips—having been deprived of normal blood circulation—attain a reduced surface temperature closer to ambient. Following this period of constriction, the occluding band can be manually loosened by pressing a button on the occluding band, thereby immediately restoring blood flow. The subsequent time-variations of the finger-tip temperature are measured by the sensor.

Where a hand mounted control unit is employed, the unit may include one or more of telemetry receiver, telemetry transmitter, data storage, battery power, digital or analog display, control buttons, timers, ambient temperature sensor, galvanic skin response (i.e., electro dermal response-EDR) monitor and a pump for controlled inflation of a finger cuff.

In one embodiment, the ring including an occluding strap or cuff also contains an additional temperature sensor that measures the ambient temperature. Both these temperature signals are digitized by a microchip-based data acquisition system which may be placed within the housing. Data is recorded for a pre-set programmable duration, sufficient to capture all relevant trends of the temperature data. Upon completion of the test, the data is transmitted to a remote telemedic computer system. The transmitted data will also contain “envelope” information identifying the device serial number, thereby identifying the human subject; several hundred simultaneous data transmissions can be handled by a dedicated telemedic computer system.

At the telemedic center, the dedicated computer system analyzes the temperature trends, and looks up relevant patient-specific information from its database. Using these inputs, a computational model calculates the DTM indices describing the functioning of the endothelial system. Physicians will thus be able to query and view various graphs and data tables and analyze the DTM indices to determine the patient's state of health. Having simultaneous access to the patient's medical history, they will be able to compare current data with past data taken under user-selectable environments. This will further allow the medical staff to take into account the various subjective environmental factors before arriving at a diagnosis. Table 1 below summarizes the salient features of this embodiment of a MDTMD according to the invention:

TABLE 1
Features of MDTMD

| | |
|---|---|
| 1 | Disposable temperature sensor probes. |
| 2 | Small and ergonomically designed device to allow for normal use |
| | of hands. |
| 3 | Use of biocompatible materials and adhesives. |
| 4 | High data storage capacity. Can store up to one week of continuous |
| | data feed. |
| 5 | Efficient wireless data transfer and management. |
| 6 | Impact proof. |
| 7 | Easy to use and removable and can be dismantled. |

At present there are no practical means to continuously monitor the cardiovascular effects of stress in ambulant subjects. Due to a lack of rigorous and sensitive methods of measuring the impact of the psychological factors on the cardiovascular system, the insidious and slowly developing symptoms of stress often go unrecognized and the effects of stress are often only recognized subsequent to severe trauma or functional disruption of the patient. At present, the cardiovascular fitness levels sufficient to tolerate stress, and the adverse short- and long-term cardiovascular effects of stress cannot be quantified. Conventional clinical assessment of cardiovascular (CV) fitness in apparently healthy subjects, such as active duty military personnel (e.g. screening for CV risk factors, exercise stress testing), fails to identify individuals with occult coronary heart disease (CHD), who are at increased near-term risk of cardiovascular events. The routine use of coronary imaging technologies (such as computer tomography, CT, heart scanning) to screen for silent CHD is cost-prohibitive, particularly in relatively young subjects.

In one embodiment of the present invention, stress monitoring by continuous skin temperature recording is combined with vasostimulation to provide individual assessments of vascular responses. In another embodiment, vascular reactivity is assessed during real-life activities as well as induced psychological stress by utilizing iontophoresis delivery of vasoactive substances combined with skin temperature monitoring of blood flow in the local acre of administration. In another embodiment, finger based cuff occlusion of the present invention implements reactive hyperemia to measure vascular reactivity by DTM and/or fingertip arterial tonometry and iontophoresis is used to detect responsiveness mediated by NO. The device is worn in ordinary conditions, normally considered to be non-stressful, to establish a “normal” individual vascular reactivity profile. The individual is then subject to various stressful conditions to determine that individual's vascular reactivity under stress.

Use of stress simulators in conjunction with ambulatory temperature recording as a monitor of an individual stress response confers several benefits. First, it allows standardization of stress conditions. Second, a series of controlled experiments with varying degrees of stress under repeatable conditions can be simulated, thereby facilitating precise measurements. Third, potentially significant variations in factors such as weather conditions, food intake and other conditions that could lead to increased measurement noise can be avoided. Use of controlled induced stress permits isolation of physiological mental stress from that of the stress experience due to physical exertion. Individuals who are more susceptible to stress can be readily identified.

Use of Ambulatory Stress and Vascular Response Monitors in Conjunction with Risk Factor Assessment: Mental stress may manifest itself in different ways in healthy young military soldiers as compared to the older war veterans or high ranking officers as well as civilians. Among sensitive individuals, the presence of inflammatory markers that promote CHD could contribute to a condition in which the slightest of the triggers due to psychological stress can be fatal. Military personnel and civilians alike include individuals who have subclinical atherosclerosis as measured by coronary artery calcium score (CACS) and carotid intima media thickness (CIMT). Certain of these individuals are more susceptible than others to psychological stress that is manifest in sympathetic nervous system vasoconstriction that is potentially life threatening. These are the individuals who are on the “fast-track” to CHD. On the other hand, the effect of stress on younger soldiers and civilians could be slightly different and will have a long-term effect on vascular health. Therefore, there is a need to identify those individuals that are classified as Very-High-Risk according to further criteria including for example those put forth in the SHAPE Task Force guidelines. See Naghavi M et al. “From Vulnerable Plaque to Vulnerable Patient: A Call for New Definitions and Risk Assessment Strategies: Part I” Circulation 108 (2003) 1664-1672; Naghavi M et al. “From Vulnerable Plaque to Vulnerable Patient: A Call for New Definitions and Risk Assessment Strategies: Part II.” Circulation 108 (2003) 1772-1778.

It is understood that variations may be made in the foregoing without departing from the scope of the disclosed embodiments. Furthermore, the elements and teachings of the various illustrative embodiments may be combined in whole or in part some or all of the illustrated embodiments. Although illustrative embodiments have been shown and described, a wide range of modification, change and substitution is contemplated in the foregoing disclosure and in some instances, some features of the embodiments may be employed without a corresponding use of other features. Accordingly, it is appropriate that the appended claims be construed broadly and in a manner consistent with the scope of the embodiments disclosed herein.

We claim:

1. A method of assessing vascular and/or neurovascular responsiveness in an individual comprising:
continuously measuring and recording skin temperature at a test location on an individual;
administering a vasostimulator compound by iontophoresis to the test location; and
determining vascular and/or neurovascular responsiveness on the basis of skin temperature changes in response to the vasoactive compound.

2. The method of claim 1, wherein the vasostimulator compound is an endothelium-dependent vasoactive compound.

3. The method of claim 2, wherein the endothelium-dependent vasostimulator compound comprises acetylcholine.

4. The method of claim 1, wherein the vasostimulator compound is an endothelium-independent vasoactive compound.

5. The method of claim 2, wherein the endothelium-independent vasostimulator compound comprises sodium nitroprusside.

6. The method of claim 1, further comprising administering a psychological stress inducer concomitant with determining responsiveness to the vasostimulator compound.

7. The method of claim 1, wherein the test location is selected from the group consisting of: forearm, wrist, forehead and finger.

8. The method of claim 1, further comprising simultaneously and continuously measuring and recording a reactive hyperemia response induced in a vascular bed feeding the test location.

9. The method of claim 1, further comprising simultaneously measuring and recording additional physiologic parameters selected from the group consisting of: pulse rate, blood pressure, galvanic response, blood oxygenation, and sweating.

10. The method of claim 1, where skin temperature is measured by a thermal potential difference using a thermocouple.

11. The method of claim 1, wherein the skin temperature is measured by an infrared detector.

12. A vascular and/or neurovascular function measurement device comprising:
a housing including an iontophoretic chamber adapted to store and deliver a charged vasostimulator e compound through skin of a patient; and
one or more temperature sensors mounted on the housing and adapted to detect changes in skin temperature in response to delivery of the vasostimulator compound.

13. The device of claim 12, wherein the temperature sensor is selected from the group consisting of: thermocouples, thermopiles and infrared detectors.

14. The device of claim 12, further comprising a heating element.

15. The device of claim 12, further comprising a chemical sensor.

16. The device of claim 12, wherein the housing is dimensioned to be affixed flat to a skin surface.

17. The device of claim 12, wherein the housing is dimension to be worn as a ring.

18. A method of vascular and/or neurovascular function measurement comprising:
initiating monitoring of an inherent temperature of a mucosal surface;
administering a vasostimulator directly to the mucosal surface;
continuing to monitor any temperature change as a consequence of administration of the vasostimulator; and
determining vascular and/or neurovascular responsiveness on the basis of temperature changes in response to the vasostimulator.

19. The method of claim 18, wherein the monitoring is by contact thermal sensor.

20. The method of claim 18, wherein the monitoring is by digital infrared thermal imaging.

21. The method of claim 18, wherein the mucosal surface is selected from the group consisting of the: eye mucosa, sublingual mucosa, intranasal mucosa, rectal mucosa, vaginal mucosal and urethral mucosa.
